(19) United States
(12) Patent Application Publication
(10) Pub. No.: US 2016/0143842 A1
(43) Pub. Date: May 26, 2016

(54) ANHYDROUS COMPOSITION WITH LIGHT TEXTURE FOR APPLICATION TO THE SKIN

(71) Applicant: Henkel AG & Co. KGaA, Duesseldorf (DE)

(72) Inventor: Alexandra Struwe, Willich (DE)

(21) Appl. No.: 15/009,466

(22) Filed: Jan. 28, 2016

Related U.S. Application Data
(63) Continuation of application No. PCT/DE2014/200218, filed on May 15, 2014.

(30) Foreign Application Priority Data
Jul. 30, 2013 (DE) 10 2013 214 843.7

(51) Int. Cl. A61K 8/92; A61K 8/29; A61Q 17/04

(52) U.S. Cl. CPC A61K 8/92; A61Q 17/04; A61K 8/29; A61K 2800/20

(57) ABSTRACT

Cosmetic compositions include, based in each case on the total weight of the composition, 0 to 3% by weight, in particular 0 to 2% by weight, of free water, one or more liquid oils in a total amount of from 40 to 80% by weight, in particular from 50 to 75% by weight, in particular from 60 to 70% by weight, at least one wax, solid dispersed in particulate form. The cosmetic composition has a viscosity of from 300 000 to 800 000 mPas, in particular from 400 000 to 600 000 mPas, has a density of from 0.95 to 1.10 g/cm3 at 20° C., has a light texture and can be spread easily on a substrate. If the substrate is skin, a pleasant skin feel is conveyed.

### FIELD OF THE INVENTION

The present invention generally relates to skin cosmetics, in particular anhydrous compositions having a particular density, which include liquid oil and solid matter dispersed in particulate form.

### BACKGROUND OF THE INVENTION

Cosmetics including solids dispersed in particulate form are known to the person skilled in the art. These are used in skin cosmetics, for example, as UV sunscreen cosmetics including titanium dioxide, as antiperspirants including particulate aluminum salts, or for decorative cosmetics as make-up including particulate color pigments in the form of powders or lipsticks. The use of solid particles for generating optical effects, such as pearlescence, is also known.

On the one hand, the stable incorporation of the solid particles into a liquid medium is problematic when formulating cosmetics including particulate solids. The particles should be incorporated so as to be storage-stable and should not settle. In cosmetics having a high oil content, furthermore the phenomenon of syneresis must be prevented. During syneresis, the composition becomes unstable, for example when pressure is applied. A liquid component of the composition then precipitates.

Sedimentation of the incorporated solid particles does not necessarily have to occur during syneresis.

Cosmetics having in particular a high content of particulate solids are often times perceived by the consumer as impractical to handle and unpleasant after application. Often times, they are difficult to spread on the substrate, for example the skin, and leave an unpleasant sensation on the substrate. Cosmetics having a high oil content are often perceived to leave an unpleasant greasy feel. The consumer lastingly feels being coated with the cosmetic. Despite the presence of the pigments, and the oils where applicable, the consumer nonetheless desires a velvety, lightweight sensation on the skin.

It is therefore desirable to provide a storage-stable cosmetic that includes dispersed particles as the solid matter and is easy to spread on a substrate, in particular on the skin. Moreover, said cosmetic is to leave a velvety and lightweight feel on the skin.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

It was found that the stated object is achieved by a viscous oil-containing and solid particle-containing composition that has a particular density, and thus a mousse-like texture. Such a cosmetic agent includes, in each case based on the total weight of the agent: 0 to 3 wt. %, in particular 0 to 2 wt. %, free water; one or more liquid oils in a total amount of 40 to 80 wt. %, in particular 50 to 75 wt. %, in particular 60 to 70 wt. %; at least one wax; solid matter dispersed in particulate form; optionally further ingredients; characterized in that the cosmetic agent has a viscosity of 300000 to 800000 mPas, in particular of 400000 to 600000 mPas (in each case measured at 20° C., Brookfield, 4 revolutions per minute, spindle TE); and the cosmetic agent has a density of 0.95 to 1.10 g/cm³ at 20° C.

A production method for such cosmetic agents is also a subject of the present invention wherein: wax is dissolved in a liquid oil phase forming a wax solution; solid matter is dispersed in particulate form in this wax solution; optionally further ingredients are added to the wax solution; optionally a) together with the solid matter in particulate form, or together with the further ingredients, or separately, at the end, at least one thickening agent for thickening oil is added, and a gaseous substance is dispersed in the resulting mixture, or during the mixing process, until the cosmetic agent has a density (measured at 20° C.) of 0.95 to 1.10 g/cm³.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject matter of the invention relates to cosmetic agents, each including, based on the total weight of the agent,
0 to 3 wt. %, in particular 0 to 2 wt. %, free water;
one or more liquid oils in a total amount of 40 to 80 wt./%;
at least one wax;
solid matter dispersed in particulate form;
optionally further ingredients;
wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.

The term “free water” shall be understood according to the invention such that the content of constitutional water, hydration water or similarly molecularly bound water that can be present in the components used, in particular in the solids dispersed in particulate form, does not constitute free water within the meaning of the present application.

All information regarding the states of matter (solid, liquid, gaseous) in this application relate to normal conditions. “Normal conditions” within the meaning of the present application refer to a temperature of 20° C. and a pressure of 1013.25 mbar. Melting point information likewise refers to a pressure of 1013.25 mbar.

It is furthermore essential that the composition according to the invention includes at least one or more liquid oils in said total amount. An oil according to the invention shall be understood to mean a liquid substance that can be mixed in bidistilled water to less than 1 wt. % under normal conditions.

In a preferred embodiment, the liquid oil is selected from at least one compound of the group consisting of liquid silicones, ester oils, trifatty acid esters of saturated and/or unsaturated, linear and/or branched C⁶ to C₂₂ fatty acids including glycerol, vegetable oils, liquid paraffin oils, isoparaffin oils, liquid synthetic hydrocarbons, liquid di-n-alkyl ethers, dicarboxylic acid esters, and symmetrical, asymmetrical or cyclic esters of carbonic acid.

It is preferred according to the invention if the agent according to the invention, based on the weight of the composition, includes liquid oil in a total amount of in particular 50 to 75 wt. %, in particular 60 to 70 wt. %, in the composition according to the invention.

The composition according to the invention preferably includes at least one silicone oil as the liquid oil.

It is preferred according to the invention if the agent according to the invention, based on the weight of the composition, includes silicone oil in a total amount of 30 to 70 wt. %, in particular of 40 to 65 wt. %, in the composition according to the invention.

The silicone oils, in turn, are preferably selected from at least one compound from the group consisting of:
(i) polyalkylsiloxanes, polyarylsiloxanes, polyalkylarylsiloxanes, which are volatile or non-volatile, straight-chain, branched or cyclic, cross-linked or not cross-linked;
(ii) polysiloxanes including, in the general structure thereof, one or more organofunctional groups, which are selected from (per)fluorinated groups;
(iii) or the mixtures thereof.

Especially particularly preferred cosmetic agents are characterized by including, as the oil, at least one silicone of formula (Si-1)

(CH₃)₃Si—[O—Si(CH₃)₂]ₓ—O—Si(CH₃)₃ (Si-1),

where x denotes a number from 0 to 200, preferably from 0 to 100, and more preferably from 0 to 20.

The preferred cosmetic agents according to the invention include at least one silicone of above formula (Si-1) as the oil. According to INCI nomenclature, these silicones are referred to as Dimethicone.

Likewise preferred silicone oils according to the invention are selected from silicones of formula (Si-2), where x is selected from integers from 1 to 20, preferably 1 to 3.

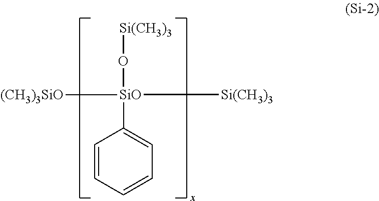

A preferred silicone oil of formula (Si-2) is available under the INCI name Phenyl Trimethicone.

It is also possible, of course, for mixtures of the above-mentioned silicones of formulas (Si-1) and (Si-2) to be present as silicone oil in the preferred agents according to the invention.

Preferred silicone oils that can be used according to the invention, in particular according to formula (Si-1), have a kinematic viscosity of 1 to 200 mm²s⁻¹, particularly preferably of 5 to 100 mm²s⁻¹, at 25° C. Such silicone oils are commercially available, for example as Dimethicone, under the trade name Xiameter PMX 200 Sil Fluid 50 CS (formerly: Dow Corning 200 fluid 500 cSt).

Within the scope of a further embodiment of the invention, preferred cosmetic agents according to the invention are those that include, as the liquid oil, at least one ester of formula (I)

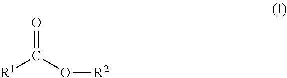

where R¹ and R², independently of one another, denote a linear or branched hydrocarbon group having 3 to 22 carbon atoms.

It is particularly preferred if, according to formula (I), R¹ is a linear or branched hydrocarbon group having 3 to 22 carbon atoms, and R² is a branched hydrocarbon group having 3 to 22 carbon atoms.

Preferred liquid oils are selected from esters according to formula (I) of C₆ to C₂₂ fatty acids (R¹=linear or branched C₅ to C₂₁ hydrocarbon) having C₃ to C₂₂ fatty alcohols (R²=linear or branched C₃ to C₂₂ hydrocarbon).

Preferred examples of fatty acid components used in the esters of formula (I) are caproic acid, caprylic acid, 2-ethyl-hexanoic acid, capric acid, lauric acid, isotridecanoic acid, myristic acid, palmitic acid, palmitoleic acid, stearic acid, isostearic acid, oleic acid, elaidic acid, petroselinic acid, linoelic acid, linolenic acid, eleostearic acid, arachyinic acid, gadoleic acid, behenic acid and erucic acid, and the mixtures thereof, such as the technical mixtures that develop, for example, in the hydrolysis of natural fats and oils under pressure, in the oxidation of aldehydes from Roelen's oxo synthesis, or the dimerization of unsaturated fatty acids. Preferred examples of the fatty alcohol components in the esters of formula (I) are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoleyl alcohol, linolenyl alcohol, eleostearyl alcohol, arachyl alcohol, gadoleyl alcohol, behenyl alcohol, erucyl alcohol, and brassidyl alcohol, and the mixtures thereof, such as the technical mixtures that develop, for example, in the high-pressure hydrogenation of technical methyl esters based on fats and oils or aldehydes from Roelen's oxo synthesis, and as monomer fraction in the dimerization of unsaturated fatty acids.

It is particularly preferred according to the invention if the compound according to formula (I) is selected from 2-ethylhexyl palmitate, 2-ethylhexyl stearate, 2-ethylhexyl cocoate, 2-ethylhexyl laurate, 2-ethylhexyl isostearate, hexyldecyl stearate, hexyldecyl laurate, isodecyl neopentanoate, isononyl isononanoate, isopropyl myristate, isopropyl palmitate, isopropyl stearate, isopropyl isostearate, isopropyl oleate, isooctyl stearate, isononanoic acid-C₁₆₋₁₈-alkyl ester, isononyl stearate, isocetyl stearate, isononyl isononanoate, isotridecyl isononanoate, cetearyl isononanoate, 2-octyldodecyl palmitate, 2-octyldodecyl myristate, 2-octyldodecyl laurate, 2-octyldodecyl stearate, butyloctanoic acid-2-butyl octanoate, coconut fatty alcohol caprinate/caprylate, n-butyl stearate, n-hexyl laurate, n-decyl oleate, cetyl oleate, oleyl oleate, oleyl erucate, erucyl oleate, erucyl erucate, or mixtures of two or more of the above-mentioned compounds.

It is particularly preferred according to the invention to select the respective hydrocarbon groups of the esters of formula (I), in particular within the scope of the preferred embodiments of said esters (vide supra), from saturated hydrocarbons.

The agents according to the invention preferably include the compounds of formula (I) in an amount from 1 to 60 wt. %, in particular from 5 to 30 wt. %, in each case based on the total weight of the agent.

Within the scope of this embodiment of the invention, preferred cosmetic agents according to the invention are again those that at least include, as the liquid oil,
at least one ester of formula (I)

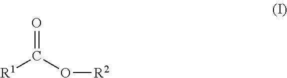

where R¹ and R², independently of one another, denote a linear or branched hydrocarbon group having 3 to 20 carbon atoms,
at least one silicone oil.

Within the scope of this combination, in turn, the preferred embodiments of the compounds of formula (I) and of the silicone oils can be used as particularly preferred components.

Within the scope of the above embodiment of the invention, particularly preferred cosmetic agents according to the invention are those that at least include, as the liquid oil,
one or more esters of formula (I),

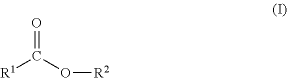

where R¹ and R², independently of one another, denote a linear or branched hydrocarbon group having 3 to 20 carbon atoms, in a total amount of 1 to 20 wt. %, in particular of 5 to 15 wt. %,
silicone oil in a total amount of 30 to 60 wt. %, in particular of 40 to 60 wt. %.

Within the scope of this combination, in turn, the preferred embodiments of the compounds of formula (I) and of the silicone oils can be used as particularly preferred components.

Triglyceride oils, such as the liquid fractions of suet, and synthetic triglycerides are also suitable as liquid oil according to the invention. Preferred triglycerides are selected from the triglycerides of linear or branched, saturated or unsaturated, optionally hydroxylated, C₈₋₃₀ fatty acids. The use of natural oils can be particularly suitable, such as soy bean oil, cottonseed oil, sunflower oil, palm oil, palm kernel oil, linseed oil, almond oil, castor oil, corn oil, rapeseed oil, olive oil, sesame oil, safflower oil, wheat germ oil, peach kernel oil, and the liquid components of coconut oil, glyceryl trioleate (triolein), capric/caprylic triglycerides, glyceryl triisostearin, glyceryl triisopalmitate, and mixtures of two or more of the aforementioned compounds.

Preferred vegetable oils are selected from at least one representative from the group consisting of amaranth oil, sunflower oil, olive oil, soy bean oil, rapeseed oil, almond oil, jojoba oil, orange oil, apricot kernel oil, macadamia nut oil, wheat germ oil, peach kernel oil, and the liquid components of coconut oil.

Preferred di-n-alkyl ethers are selected from di-n-alkyl ethers having in total between 12 and 36 carbon atoms, in particular 12 to 24 carbon atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether, and di-tert-butyl ether, di-iso-pentyl ether, di-3-ethyldecyl ether, tert-butyl-n-octyl ether, iso-pentyl-n-octyl ether and 2-methyl-pentyl-n-octyl ether. The compounds 1,3-bis(2-ethylhexyl)cyclohexane (Cetiol® S) and di-n-octyl ether Cetiol® OE) available as commercial products may be preferred.

Preferred dicarboxylic acid esters are selected from at least one compound of the group consisting of di-n-butyl adipate, di-(2-ethylhexyl) adipate, di-(2-ethylhexyl) succinate and diisotridecyl acelaate, and diol esters such as ethylene glycol dioleate, ethylene glycol diisotridecanoate, propylene glycol di(2-ethylhexanoate), propylene glycol diisostearate, propylene glycol dipelargonate, butanediol diisostearate, and neopentyl glycol dicaprylate.

Natural and synthetic hydrocarbons, such as paraffin oils, C₁₈ to C₃₀ isoparaffins, in particular isoeicosane, polyisobutene or polydecene, which are available under the designation Emery® 3004, 3006, 3010 or under the designation Ethylflo from Albemarle or Nexbase® 2004G from Nestle, for example, and 1,3-bis(2-ethylhexyl)cyclohexane (available under the trade name Cetiol® S from Cognis, for example) are likewise among the liquid oils that can preferably be used according to the invention.

Further preferred liquid oils according to the invention are selected from the benzoic acid esters of linear or branched C₈₋₃₀ alkanols. Particularly preferred are benzoic acid-C₁₂-C₁₅-alkyl esters, for example available as the commercial product Finsolv® TN, benzoic acid isostearyl esters, for example available as the commercial product Finsolv® SB, ethylhexyl benzoate, for example available as the commercial product Finsolv® EB, and benzoic acid 2-octyldodecyl esters, for example available as the commercial product Finsolv® BOD.

Further preferred liquid oils according to the invention are selected from the dicarboxylic acid esters of linear or branched C₂ to C₁₀ alkanols, in particular diisopropyl adipate, di-n-butyl adipate, di-(2-ethylhexyl) adipate, dioctyl adipate, diethyl-/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl) succinate.

Further preferred liquid oils according to the invention are selected from the symmetrical, asymmetrical or cyclic esters of carbonic acid comprising alkanols, such as glycerol carbonate, di-n-caprylyl carbonate (Cetiol® CC) or the esters according to the teaching of DE 19756454 A1.

It is furthermore essential that the agents according to the invention include at least one wax. Waxes are generally understood to mean solids that are reaction products of carboxylic acids and alcohols. These reaction products in the form of wax are solid to brittle-hard, kneadable up to 20° C., and melt at 30° C. to 90° C.

Waxes according to the invention preferably have a melting point in a range from 30° C. to 60° C., particularly preferably in a range from 30° C. to 50° C., especially particularly preferably in a range from 30° C. to 40° C.

It is preferred according to the invention if at least half the amount of the wax that is present (particularly preferably at least 75% of the amount of the wax that is present) is present in dissolved form in the liquid oil of the cosmetic agent according to the invention. When, in connection with this embodiment, at least one ester of formula (I) (vide supra) is present in the liquid oil component, it is preferred according to the invention within the scope of the production process to dissolve wax in an amount of at least one ester of formula (I) (vide supra), and then incorporate it in this dissolved form into the cosmetic agent according to the invention.

It is especially particularly preferred according to the invention if the agent according to the invention includes at least one wax, selected from at least one wax of the group of waxes having the INCI names Cocoglycerides, Cetyl Palmitate, Myristyl Myri state.

The agents according to the invention preferably include wax in a total amount of 0.05 to 8.0 wt. %, particularly preferably of 0.1 to 5.0 wt. %, and especially particularly preferably of 0.5 to 3.0 wt. %, in each case based on the total weight of the agent.

It is essential that the cosmetic agents according to the invention include solid matter dispersed in particulate form. Within the meaning of the invention, particles are particles (see DIN 66160:1992-09) of solids which are present as grains. Consequently, particles of solids which are present as grains are present in the agent according to the invention in dispersed form.

The particles preferably have a mean particle diameter (volume mean) of 0.01 to 3.0 μm, in particular of 0.05 to 1.0 μm, especially particularly preferably of 0.1 to 0.5 μm.

According to the invention, preferred cosmetic agents are those that include solid matter dispersed in particulate form in a total amount of 10 to 30 wt. %.

In preferred cosmetic agents according to the invention, at least one inorganic solid, in particular at least one metal oxide, is present as the solid matter dispersed in particulate form. The metal oxides, in turn, are preferably selected from aluminates, aluminum silicates, titanium dioxide, zinc oxide, iron oxides, tin dioxide, nacreous pigments, and mixtures of two or more of the aforementioned metal oxides.

Frequently used nacreous pigments include mother of pearl (made of ground shells), monocrystalline nacreous pigments, such as bismuth oxychloride, and nacreous pigments based on mica, mica/metal oxide, or titanium dioxide/metal oxide. The mixed nacreous pigments mentioned last are provided with a metal oxide coating that is different from the particulate material of the core. Through the use of nacreous pigments, color effects and/or brilliance are achieved in the agents according to the invention or on the substrate treated therewith.

Particularly preferred aluminates are selected from active aluminum oxide, alpha-aluminum oxide, beta-aluminum oxide, gamma-aluminum oxide, and the mixtures thereof.

Particularly preferred aluminum silicates (also referred to as aluminosilicates) are selected from phyllosilicates, tectosilicates.

Particularly suitable phyllosilicates are selected from kaolins (here, in particular from kaolinite, dickite, halloysite and nacrite), serpentine, talcum, pyrophyllite, montmorrillonite, quartz, bentonite, mica (here, in particular from illite, muscovite, paragonite, phlogopite, biotite, lepidolite, margarite, smectite (here, in particular from montmorrillonite, saponite, nontronite, hectorite)).

Preferably suitable tectosilicates are selected from feldspar minerals (in particular albite, orthoclase, anorthite, leucite, sodalite, hauyne, labradorite, lasurite, nosean, nepheline).

Preferred titanium dioxides are those distributed under the trade name Kronos by Kronos, in particular Kronos 1171.

Preferred iron oxides are Fe₂O₃, for example having INCI CI 77491, in particular commercially as Unipur Red LC 281 EM® by Sensient, Fe₂O₃.nH₂O, for example having INCI CI 77491, in particular commercially as Unipur Red LC 281 EM® by Sensient, FeO.Fe₂O₃, for example having INCI CI 77499, in particular commercially as Unipur Black LC 989 EM® by Sensient.

Preferred cosmetic agents according to the invention are characterized in that they (together with aforementioned inorganic solids, or without the presence of aforementioned inorganic solids as solid matter dispersed in particulate form) include at least one starch as the solid matter dispersed in particulate form.

Starch is a reserve carbohydrate that is stored in many plants in the form of usually 1 to 200 μm large starch grains (granules) in various parts of the plants, such as in bulbs or roots, grain seeds, fruits, and in the marrow. Starch belongs to the family of homoglycans and is a polycondensation product of D-glucose. Starch is composed of three structurally different polymers of d-glucopyranose, namely amylose, amylopectin and what is known as an intermediate fraction. Higher plants include 0 to 45 wt. %, based on the dry substance. The intermediate fraction, which is also referred to as abnormal amylopectin, is between the amylosis and the amylopectin from a structural perspective. The quantity information for amylopectin defined within the scope of the present application include the intermediate fraction. Amylose is predominantly composed of a linear chain of D-glucose units linked by α-1,4-glycosidic bonds, Mr 50000 to 150000. The resulting chains form double helices in the starch. In addition to the α-1,4 bonds described for amylose, amylopectin also includes α-1,6 bonds as branching points in an amount from 4 to 6%. The average distance between the branching points is approximately 12 to 17 glucose units. The molar mass of 107 to 7·108 corresponds to approximately 105 glucose units, whereby amylopectin belongs to the largest biopolymers. Said branching points are distributed across the molecule in such a way that a cluster structure having relatively short side chains develops. Two of these side chains in each case form a double helix. As a result of the many branching points, amylopectin has relatively good solubility in water.

A starch that can preferably be used according to the invention is selected from at least one polycondensation product of D-glucose, obtained from the starch of potatoes, corn, rice, peas, acorns, chestnuts, wheat, bananas, sago, millet, sorghum, oats, barley, rye, beans, batata, arrowroot or tapioca. It is particularly preferred if the agent according to the invention includes at least one starch which is tapioca starch, potato starch, corn starch or rice starch. Mixtures of the aforementioned starch compounds are also covered according to the invention. Tapioca starch is especially particularly preferred.

The starch compound is preferably present in the agents according to the invention in amounts of 0.05 to 8.0 wt. %, and in particular of 0 to 7.0 wt. %, in each case based on the weight of the agent.

It is preferred according to the invention within the scope of a further embodiment of the invention if the solid matter dispersed in particulate form is coated on the surface of the solid matter particle with at least one water-in-oil emulsifier.

Particularly suitable water-in-oil emulsifiers are preferably selected from at least one compound from the group consisting of:
partial esters of polyglycerols having n=2 to 10 glycerol units and esterified with 1 to 5 saturated or unsaturated, linear or branched, optionally hydroxylated C₈ to C₃₀ fatty acid esters;
linear or branched, saturated or unsaturated C₁₂ to C₃₀ alkanols, each being etherified with 1 to 4 ethylene oxide units per molecule, which are exceptionally preferably selected from steareth, ceteth, myristeth, laureth, trideceth, arachideth and beheneth, each having 1 to −4 ethylene oxide units per molecule, in particular Steareth-2, Steareth-3, Steareth-4, Ceteth-2, Ceteth-3, Ceteth-4, Myristeth-2, Myristeth-3, Myristeth-4, Laureth-2, Laureth-3, Laureth-4, Trideceth-2, Trideceth-3 and Trideceth-4, and mixtures thereof;
linear saturated alkanols comprising 12 to 30 carbon atoms, in particular comprising 16 to 22 carbon atoms, in particular cetyl alcohol, stearyl alcohol, arachidyl alcohol, behenyl alcohol, and lanolin alcohol, or mixtures of these alcohols, as they are obtainable from the technical hydrogenation of vegetable and animal fatty acids;
esters, and in particular partial esters, made of a polyol having 2 to 6 carbon atoms, and linear saturated and unsaturated fatty acids having 12 to 30, in particular 14 to 22, carbon atoms, which can be hydroxylated. Such esters or partial esters are, for example, the monoesters and diesters of glycerol or ethylene glycol, or the monoesters of propylene glycol having linear saturated and unsaturated C₁₂ to C₃₀ carboxylic acids, which can be hydroxylated, in particular those comprising palmitic and stearic acid, the sorbitan monoesters, diesters or triesters of linear saturated and unsaturated C₁₂ to C₃₀ carboxylic acids, which can be hydroxylated, in particular those of myristic acid, palmitic acid, stearic acid, or mixtures of these fatty acids, the pentaerythrityl monoesters, diesters, triesters and tetraesters, and the methyl glucose monoesters and diesters of linear, saturated and unsaturated C₁₂ to C₃₀ carboxylic acids, which can be hydroxylated, of which the monoesters, diesters, triesters and tetraesters of pentaerythritol comprising linear saturated fatty acids having 12 to 30, in particular 14 to 22, carbon atoms, which can be hydroxylated, and mixtures thereof, are particularly preferred as stabilizers and/or water binders. The monoesters and diesters are particularly preferred according to the invention. Preferred C₁₂ to C₃₀ fatty acid groups according to the invention are selected from lauric acid, myristic acid, palmitic acid, stearic acid, arachinic acid and behenic acid groups, the stearic acid group being particularly preferred;
sterols, which is to say steroids, which carry a hydroxyl group at the C3 atom of the steroid skeleton and are isolated both from animal tissue (zoosterols, such as cholesterol, lanosterol), from plants (phytosterols, such as ergosterol, stigmasterol, sitosterol) and from fungi and yeasts (mycosterols) and which may be low-ethoxylated (1 to 5 EO);
alkanols and carboxylic acids, each having 8 to 24 C atoms, in particular having 16 to 22 C atoms, in the alkyl groups and 1 to 4 ethylene oxide units per molecule, which have an HLB value of greater than 1.0 and/or smaller than/equal to 7.0;
glycerol monoethers of saturated and/or unsaturated, branched and/or unbranched alcohols having a chain length of 8 to 30, in particular 12 to 18 carbon atoms.

Liquid partial esters of polyglycerols having n=2 to 10 glycerol units and esterified with 1 to 5 saturated or unsaturated, linear or branched, optionally hydroxylated C₈ to C₃₀ fatty acid esters are particularly preferred water-in-oil emulsifiers that are available according to the invention. It is especially particularly preferred if the water-in-oil emulsifier is selected from at least one emulsifier that comprises 2 to 6 glycerol units bonding covalently to each other and at least one (in particular branched) alkyl group having 8 to 20 carbon atoms.

It is essential that the agents according to the invention have a viscosity of 300000 to 800000 mPas, in particular of 400000 to 600000 mPas (in each case measured at 20° C., Brookfield, 4 revolutions per minute, spindle TE).

These viscosity levels can also be set without the addition of an additional thickening agent for thickening oil. If this should not be the case, it is recommended to add a thickening agent for thickening oil to the formulation. Further preferred cosmetic agents according to the invention are therefore characterized by additionally including at least one thickening agent for thickening oil. These additional thickening agents for thickening oil are different from the above-mentioned components, whose presence in the agent according to the invention is essential.

It is expedient according to the invention that, if the additional thickening agents for thickening oil are used in the cosmetic agent according to the invention, said thickening agents are added only in such an amount until the viscosity is in the viscosity range according to the invention.

Preferred thickening agents according to the invention for thickening oil are selected from hydrophobized clay minerals, silica (in particular fumed silica), ethylene/propylene/styrene copolymers, butylene/ethylene/styrene copolymers, dextrin esters and/or silicone elastomers.

Preferred hydrophobized clay minerals are selected from hydrophobized montmorrillonites, hydrophobized hectorites and hydrophobized bentonites, particularly preferably made of disteardimonium hectorite, stearalkonium hectorite, quaternium-18 hectorite, and quaternium-18 bentonite. The commercially available thickening agents provide these hydrophobized clay minerals in the form of a gel in an oil component, preferably in cyclomethicone and/or a non-silicone oil component, such as propylene carbonate. Such gels are available, for example, under the trade name Bentone® or thixogel.

Preferred compositions according to the invention are characterized by including hydrophobized clay mineral in a total amount of 0.5 to 10 wt. %, preferably 1 to 7 wt. %, particularly preferably 2 to 6 wt. %, exceptionally preferably 3 to 5 wt. %, in each case based on the total weight of the composition according to the invention.

Further especially particularly preferred thickening agents according to the invention for thickening oil are selected from silica, in particular fumed silica (such as the commercial products of the Aerosil® series from Evonik Degussa). These may be hydrophobized on the surface thereof by way of chemical modification (such as the silylated silica having the INCI name Silica Silylate); however, this is not preferred. Particularly preferably suitable fumed silica according to the invention has the INCI name Silica.

If fumed silica is additionally used as a thickening agent for thickening oil, particularly storage-stable cosmetic agents of the present invention are obtained.

Preferred cosmetic agents according to the invention are characterized by including silica, preferably fumed silica, in a total amount of 0.1 to 6 wt. %, preferably 0.5 to 5 wt. %, particularly preferably 1.0 to 4.5 wt. %, especially particularly preferably 1.5 to 3.0 wt. %, exceptionally preferably 1.9 to 2.6 wt. %, in each case based on the total weight of the composition according to the invention.

Further lipophilic thickening agents that can be used according to the invention are selected from ethylene/propylene/styrene copolymers and butylene/ethylene/styrene copolymers. The copolymers are particularly preferably used as a pre-thickened oil-based gel. Such gels are available, for example, under the trade name Versagel® (ex Penreco). Gels including mineral oil, hydrogenated polyisobutene, isoparaffins such as isohexadecane or isododecane, and including ester oils, in particular isopropyl palmitate or isopropyl myristate, are preferred.

Further lipophilic thickening agents that can be used according to the invention are selected from silicone elastomers. A further preferred embodiment of the invention is characterized by including at least one silicone elastomer, obtainable by cross-linking an organopolysiloxane that includes at least 2 C₂ to C₁₀ alkenyl groups having a terminal double bond in each molecule with an organopolysiloxane that includes at least 2 silicone-bonded hydrogen atoms in each molecule.

Particularly preferred organopolysiloxanes according to the invention comprising at least 2 C₂ to C₁₀ alkenyl groups having a terminal double bond in the molecule are selected from methylvinylsiloxanes, methylvinylsiloxane-dimethylsiloxane copolymers, dimethylpolysiloxanes including dimethylvinylsiloxy end groups, dimethylsiloxane-methylphenylsiloxane copolymers including dimethylvinylsiloxy end groups, dimethylsiloxane-diphenylsiloxane-methylvinylsiloxane copolymers including dimethylvinylsiloxy end groups, dimethylsiloxane-methylvinylsiloxane copolymers including trimethylsiloxy end groups, dimethylsiloxane-methylphenylsiloxane-methylvinylsiloxane copolymers including trimethylsiloxy end groups, methyl-(3,3,3-trifluoropropyl)polysiloxanes including dimethylvinylsiloxy end groups, and dimethylsiloxane-methyl-(3,3,3-trifluoropropyl)-siloxane copolymers including dimethylvinylsiloxy end groups.

Particularly preferred cross-linking organopolysiloxanes according to the invention comprising at least two silicone-bonded hydrogen atoms are selected from methyl hydrogen polysiloxanes including trimethylsiloxy end groups, dimethylsiloxane-methyl hydrogen siloxane copolymers including trimethylsiloxy end groups, and cyclic dimethylsiloxane-methylhydrogen-siloxane copolymers.

Particularly preferred silicone elastomers according to the invention, which, as raw material, are present already pre-swelled in a silicone that is liquid at room temperature under normal conditions and represent a silicone-based gel, are commercially available, for example under the trade name Corning 9040 Silicone Elastomer Blend (a cyclomethicone (and) dimethicone crosspolymer from Dow Corning; silicone elastomer content 12 to 13 wt. %), SFE 168, a cyclomethicone (and) dimethicone/vinyl dimethicone crosspolymer from GE Silicones, vinyl dimethicone crosspolymers, included in KSG-15 (cyclomethicone (and) dimethicone/vinyl dimethicone crosspolymer, silicone elastomer content 4 to 10 wt. %), KSG-16 (dimethicone (and) dimethicone/vinyl dimethicone crosspolymer, silicone elastomer content 20 to 30 wt. %), KSG-17 (cyclomethicone (and) dimethicone/vinyl dimethicone crosspolymer), KSG-18 (phenyl trimethicone (and) dimethicone/phenyl vinyl dimethicone crosspolymer, silicone elastomer content 10 to 20 wt. %); and KSG-20, available from Shin Etsu Silicones of America (Akron, Ohio), and from Grant Industries Inc. (Elmwood Park, N.J.), the products from the Gransil® series, in particular Gransil SR-CYC (cyclomethicone and stearyl-vinyl/hydromethylsiloxane copolymer), Gransil® RPS Gel (INCI name: Cyclopentasiloxane and Polysilicone-11), Gransil® GCM-4 (INCI name: Cyclotetrasiloxane and Polysilicone-11), Gransila GCM-5 (INCI name: Cyclopentasiloxane and Polysilicone-11), Gransil® RPS (INCI name: Cyclopentasiloxane and Polysilicone-1), GI-CD 10 (INCI name: Cyclopentasiloxane (and) Stearoxymethicone/Dimethicone Copolymer (and) Dimethicone), Gransil® IDS (INCI name: Isododecane (and) Cyclotetrasiloxane (and) Polysilicone-11), Gransil® PC-12 (INCI name: Isododecane (and) Polysilicone-11), Gransil®IDS-5 (INCI name: Isododecane (and) Cyclopentasiloxane (and) Polysilicone-11), Gransil APK-1 (INCI name: Dimethicone and Cyclopentasiloxane and Polysilicone-11 and Nylon-12 and Methyl Methacrylate/Acrylonitrile Copolymer and PEG-10 Dimethicone and Polysorbate-40 and Isohexadecane and Ammonium Polyacryloyldimethyl Taurate), Gransil® DMCM-5 (INCI name: Dimethicone and Cyclopentasiloxane and Polysilicone-11), Gransil® DMG-6 with dimethicone (6 cSt) (INCI name: Dimethicone and Polysilicone-11), Gransil® DMG-20 with dimethicone (20 cSt) (INCI name: Dimethicone and Polysilicone-11), Gransil® AM-8 Gel (INCI name: Caprylyl Methicone and Cyclopentasiloxane and Polysilicone-11), Gransil® DM 5 with dimethicone (5 cSt) (INCI name: Dimethicone and Polysilicone-11), Gransil® DMID (INCI name: Dimethicone and Isododecane and Polysilicone-11), Gransil® PM (INCI name: Phenyl Trimethicone and Polysilicone-11), Gransil® ININ (INCI name: Isononyl Isononanoate (and) Polysilicone-11).

Silicone elastomers which, as raw material, are present already pre-swelled in a silicone oil, fat or wax that is liquid at room temperature under normal conditions and represent a silicone-based/non-silicone-based gel, are likewise preferably used in the compositions according to the invention. Such silicone elastomer compositions are likewise commercially available, for example under the trade name Gransil® MLB (INCI name: Cyclopentasiloxane and Polysilicone-11 and Beeswax), Gransil® PS (INCI name: Cyclotetrasiloxane and Polysilicone-11 and Petrolatum), Gransil® PS-5 (INCI name: Cyclopentasiloxane and Polysilicone-11 and Petrolatum), Gransil® DMG-20 P with dimethicone (20 cSt) and petrolatum (INCI name: Dimethicone and Polysilicone-11 and Petrolatum), Gransil® RJO (INCI name: Cyclopentasiloxane and Polysilicone-11 and Jojoba Oil), Gransil® LANO (INCI name: Cyclopentasiloxane and Polysilicone-11 and Lanolin), Gransil® OHS-5 (INCI name: Cyclopentasiloxane and Polysilicone-11 and Octyl Hydroxystearate), and Gransil DML (INCI name: Dimethicone (and) Neopentyl Glycol Diheptanoate (and) Polysilicone-11).

A further preferred embodiment of the invention is characterized in that the silicone elastomer can be obtained by cross-linking an organopolysiloxane that includes at least 2 C₂ to C₁₀ alkenyl groups having a terminal double bond in each molecule with at least one alpha,omega-diene. Particularly preferred cross-linking alpha,omega-dienes according to the invention have the formula CH₂═CH(CH₂)ₓCH═CH₂, where x=1 to 20. Particularly preferred alpha,omega-dienes are selected from 1,4-pentadiene, 1,5-hexadiene, 1,6-heptadiene, 1,7-octadiene, 1,8-nonadiene, 1,11-dodecadiene, 1,13-tetradecadiene, and 1,19-eicosadiene.

Preferred compositions according to the invention are characterized by including at least one silicone elastomer in a total amount of 0.05 to 3 wt. %, preferably 0.1 to 2 wt. %, particularly preferably 0.2 to 1.0 wt. %, exceptionally preferably 0.3 to 0.5 wt. %, in each case based on the total weight of the composition according to the invention.

Oil-based compositions that include particulate solids typically have a density of greater than 1.2 g/cm³ at 20° C. The agents according to the invention have a density of 0.95 to 1.10 g/cm³ at 20° C. The density of the agents according to the invention is consequently significantly lower than in customary comparable compositions of the related art.

A person skilled in the art knows reliable measuring methods for reproducibly and unambiguously determining the density. Within the scope of the present application, the density was determined by way of a digital density meter (such as Chempro DMA 4100M or Mettler-Toledo Density Meter DM40). The measuring principle is based on what is known as the flexural vibration method, in which the substance is filled into a U-shaped glass tube, which is open at the ends, of the measuring device. Said glass tube is maintained at a constant temperature (20° C. here), caused to oscillate electronically, and the natural frequency of the oscillation is determined. This natural frequency is characteristic of the density of the sample.

Lowering of the density is particularly preferably achieved by way of dispersion of gas. Particularly preferred cosmetic agents according to the invention therefore include dispersed gas.

Preferably suitable gases according to the invention are air, nitrogen, oxygen, carbon dioxide, argon, nitrous oxide (air being particularly preferred).

For example, gas can be incorporated into the cosmetic agent according to the invention by way of injection, recirculation, extrusion or stirring. The gas component is particularly preferably dispersed into the cosmetic agent according to the invention by way of a homogenizer.

A particularly preferred embodiment of the invention is characterized by the following items:
(A): A cosmetic agent, including in each case based on the total weight of the agent:
   0 to 3 wt. %, in particular 0 to 2 wt. %, free water;
   one or more liquid oils in a total amount of 40 to 80 wt. %, in particular 50 to 75 wt. %, in particular 60 to 70 wt. %;
   at least one wax having a melting point in the range from 30° C. to 60° C., particularly preferably in a range from 30° C. to 50° C., especially particularly preferably in a range from 30° C. to 40° C.;
   solid matter dispersed in particulate form;
   optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.
(B): A cosmetic agent, including in each case based on the total weight of the agent:
   0 to 3 wt. %, in particular 0 to 2 wt. %, free water;
   one or more liquid oils in a total amount of 40 to 80 wt. %, in particular 50 to 75 wt. %, in particular 60 to 70 wt. %;
   at least one wax having a melting point in the range from 30° C. to 60° C., particularly preferably in a range from 30° C. to 50° C., especially particularly preferably in a range from 30° C. to 40° C.;
   solid matter dispersed in particulate form;
   optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.
(C): A cosmetic agent, including in each case based on the total weight of the agent:
   0 to 3 wt. %, in particular 0 to 2 wt. %, free water;
   one or more liquid oils in a total amount of 40 to 80 wt. %, in particular 50 to 75 wt. %, in particular 60 to 70 wt. %;
   at least one wax having a melting point in the range from 30° C. to 60° C., particularly preferably in a range from 30° C. to 50° C., especially particularly preferably in a range from 30° C. to 40° C.;
   solid matter dispersed in particulate form in a total amount of 10 to 30 wt. %;
   optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.
(D): A cosmetic agent, including in each case based on the total weight of the agent:
   0 to 3 wt. %, in particular 0 to 2 wt. %, free water;
   one or more liquid oils in a total amount of 40 to 80 wt. %, in particular 50 to 75 wt. %, in particular 60 to 70 wt. %;
   at least one wax having a melting point in a range from 30° C. to 60° C., particularly preferably in a range from 30° C. to 50° C., especially particularly preferably in a range from 30° C. to 40° C., in a total amount of 0.05 to 8.0 wt. %, particularly preferably of 0.1 to 5.0 wt. %, and especially particularly preferably of 0.5 to 3.0 wt. %;
   solid matter dispersed in particulate form;
   optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.
(E): A cosmetic agent, including in each case based on the total weight of the agent:
   0 to 3 wt. %, in particular 0 to 2 wt. %, free water;
   one or more liquid oils in a total amount of 40 to 80 wt. %, in particular 50 to 75 wt. %, in particular 60 to 70 wt. %, wherein, at least the following is present as the liquid oil:
      at least one ester of formula (I),

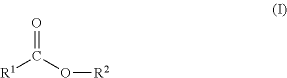

where R¹ and R², independently of one another, denote a linear or branched hydrocarbon group having 3 to 20 carbon atoms,
at least one silicone oil;
at least one wax having a melting point in the range from 30° C. to 60° C., particularly preferably in a range from 30° C. to 50° C., especially particularly preferably in a range from 30° C. to 40° C.;
solid matter dispersed in particulate form;
optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.
(F): A cosmetic agent, including in each case based on the total weight of the agent:
   0 to 3 wt. %, in particular 0 to 2 wt. %, free water;
   one or more liquid oils in a total amount of 40 to 80 wt. %, in particular 50 to 75 wt. %, in particular 60 to 70 wt. %, wherein, at least the following is present as the liquid oil:
      at least one ester of formula (I),

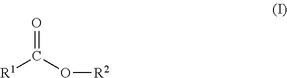

where R¹ and R², independently of one another, denote a linear or branched hydrocarbon group having 3 to 20 carbon atoms,
at least one silicone oil;
at least one wax having a melting point in the range from 30° C. to 60° C., particularly preferably in a range from 30° C. to 50° C., especially particularly preferably in a range from 30° C. to 40° C.;
solid matter dispersed in particulate form;
optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.
(G): A cosmetic agent, including in each case based on the total weight of the agent:
   0 to 3 wt. %, in particular 0 to 2 wt. %, free water;
   one or more liquid oils in a total amount of 40 to 80 wt. %, in particular 50 to 75 wt. %, in particular 60 to 70 wt. %, wherein, at least the following is present as the liquid oil:
      at least one ester of formula (I),

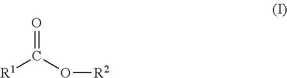

where R¹ and R², independently of one another, denote a linear or branched hydrocarbon group having 3 to 20 carbon atoms,
at least one silicone oil;
at least one wax having a melting point in the range from 30° C. to 60° C., particularly preferably in a range from 30° C. to 50° C., especially particularly preferably in a range from 30° C. to 40° C.;
solid matter dispersed in particulate form in a total amount of 10 to 30 wt. %;
optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.
(H): A cosmetic agent, including in each case based on the total weight of the agent:
   0 to 3 wt. %, in particular 0 to 2 wt. %, free water;
   one or more liquid oils in a total amount of 40 to 80 wt. %, in particular 50 to 75 wt. %, in particular 60 to 70 wt. %, wherein, at least the following is present as the liquid oil:
      at least one ester of formula (I),

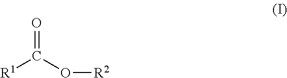

where R¹ and R², independently of one another, denote a linear or branched hydrocarbon group having 3 to 20 carbon atoms,
at least one silicone oil;
at least one wax having a melting point in a range from 30° C. to 60° C., particularly preferably in a range from 30° C. to 50° C., especially particularly preferably in a range from 30° C. to 40° C., in a total amount of 0.05 to 8.0 wt. %, particularly preferably of 0.1 to 5.0 wt. %, and especially particularly preferably of 0.5 to 3.0 wt. %;
solid matter dispersed in particulate form;
optionally further ingredients;

wherein
the cosmetic agent has a viscosity from 300000 to 800000 mPas, in particular from 400000 to 600000 mPas (in each case measured at 20° C., Brookfield, 4 revolutions per minute, spindle TE); and
the cosmetic agent has a density of 0.95 to 1.10 g/cm³ at 20° C.
(I): A cosmetic agent, including in each case based on the total weight of the agent:
   0 to 3 wt. %, in particular 0 to 2 wt. %, free water;
   one or more liquid oils in a total amount of 40 to 80 wt. %, in particular 50 to 75 wt. %, in particular 60 to 70 wt. %, wherein, at least the following is present as the liquid oil:
      one or more esters of formula (I),

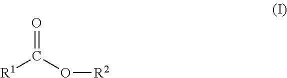

where R¹ and R², independently of one another, denote a linear or branched hydrocarbon group having 3 to 20 carbon atoms, in a total amount of 1 to 20 wt. %, in particular of 5 to 15 wt. %,
silicone oil in a total amount of 30 to 60 wt. %, in particular of 40 to 60 wt. %/o;
at least one wax having a melting point in the range from 30° C. to 60° C., particularly preferably in a range from 30° C. to 50° C., especially particularly preferably in a range from 30° C. to 40° C.;
solid matter dispersed in particulate form;
optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.
(J): A cosmetic agent, including in each case based on the total weight of the agent:
   0 to 3 wt. %, in particular 0 to 2 wt. %, free water;
   one or more liquid oils in a total amount of 40 to 80 wt. %, in particular 50 to 75 wt. %, in particular 60 to 70 wt. %, wherein, at least the following is present as the liquid oil:
      one or more esters of formula (I),

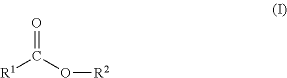

where R¹ and R², independently of one another, denote a linear or branched hydrocarbon group having 3 to 20 carbon atoms, in a total amount of 1 to 20 wt. %, in particular of 5 to 15 wt. %,
silicone oil in a total amount of 30 to 60 wt. %, in particular of 40 to 60 wt. %;
at least one wax having a melting point in the range from 30° C. to 60° C., particularly preferably in a range from 30° C. to 50° C., especially particularly preferably in a range from 30° C. to 40° C.;
solid matter dispersed in particulate form;
optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.
(K): A cosmetic agent, including in each case based on the total weight of the agent:
   0 to 3 wt. %, in particular 0 to 2 wt. %, free water;
   one or more liquid oils in a total amount of 40 to 80 wt. %, in particular of 50 to 75 wt. %, in particular of 60 to 70 wt. %, wherein, at least the following is present as the liquid oil:
      one or more esters of formula (I),

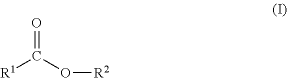

where R¹ and R², independently of one another, denote a linear or branched hydrocarbon group having 3 to 20 carbon atoms, in a total amount of 1 to 20 wt. %, in particular of 5 to 15 wt. %, and
silicone oil in a total amount of 30 to 60 wt. %, in particular of 40 to 60 wt. %;
at least one wax having a melting point in the range from 30° C. to 60° C., particularly preferably in a range from 30° C. to 50° C., especially particularly preferably in a range from 30° C. to 40° C.;
solid matter dispersed in particulate form in a total amount of 10 to 30 wt. %;
optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.
(L): A cosmetic agent, including in each case based on the total weight of the agent:
   0 to 3 wt. %, in particular 0 to 2 wt. %, free water;
   one or more liquid oils in a total amount of 40 to 80 wt. %, in particular of 50 to 75 wt. %, in particular of 60 to 70 wt. %, wherein, at least the following is present as the liquid oil:
      one or more esters of formula (I),

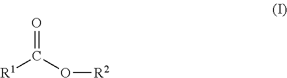

where R¹ and R², independently of one another, denote a linear or branched hydrocarbon group having 3 to 20 carbon atoms, in a total amount of 1 to 20 wt. %, in particular of 5 to 15 wt. %, and
silicone oil in a total amount of 30 to 60 wt. %, in particular of 40 to 60 wt. %;
at least one wax having a melting point in a range from 30° C. to 60° C., particularly preferably in a range from 30° C. to 50° C., especially particularly preferably in a range from 30° C. to 40° C., in a total amount of 0.05 to 8.0 wt. %, particularly preferably of 0.1 to 5.0 wt. %, and especially particularly preferably of 0.5 to 3.0 wt. %;
solid matter dispersed in particulate form;
optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.
(M): A cosmetic agent, including in each case based on the total weight of the agent:
   0 to 3 wt. %, in particular 0 to 2 wt. %, free water;
   one or more liquid oils in a total amount of 50 to 75 wt. %, in particular 60 to 70 wt. %, wherein at least the following is present as the liquid oil:
      one or more esters of formula (I),

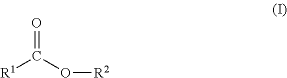

where R¹ and R², independently of one another, denote a linear or branched hydrocarbon group having 3 to 20 carbon atoms, in a total amount of 1 to 15 wt. %, in particular of 5 to 15 wt. %, and
   silicone oil in a total amount of 30 to 60 wt. %, in particular of 40 to 60 wt. %;
at least one wax having a melting point in a range from 30° C. to 60° C., particularly preferably in a range from 30° C. to 50° C., especially particularly preferably in a range from 30° C. to 40° C., in a total amount of 0.05 to 8.0 wt. %, particularly preferably of 0.1 to 5.0 wt. %, and especially particularly preferably of 0.5 to 3.0 wt. %;
solid matter dispersed in particulate form in a total amount of 10 to 30 wt. %;
optionally further ingredients;

wherein
the cosmetic agent has a viscosity of 300000 to 800000 mPas, in particular of 400000 to 600000 mPas (in each case measured at 20° C., Brookfield, 4 revolutions per minute, spindle TE), and
the cosmetic agent has a density of 0.95 to 1.10 g/cm³ at 20° C.

It is preferred according to the invention if each of the embodiments (A) through (M) additionally includes at least one thickening agent for thickening oil (in particular silica, particularly preferably fumed silica).

A second subject matter relates to a production method for cosmetic agents, in which
wax is dissolved in a liquid oil phase forming a wax solution;
solid matter in particulate form is dispersed in this wax solution;
optionally further ingredients are added to the wax solution;
optionally a) together with the solid matter in particulate form, or
   b) together with the further ingredients, or
   c) separately, at the end,
   at least one thickening agent for thickening oil is added,
      and a gaseous substance is dispersed in the resulting mixture, or during the mixing process, until the cosmetic agent has a density (measured at 20° C.) of 0.95 to 1.10 g/cm³.

For example, the gaseous substance can be incorporated into the cosmetic agent according to the invention by way of injection, recirculation, extrusion or stirring. The gaseous substance is particularly preferably dispersed into the cosmetic agent according to the invention using a homogenizer.

Preferably suitable gaseous substances according to the invention are air, nitrogen, oxygen, carbon dioxide, argon, nitrous oxide, or mixtures of two or more of the aforementioned gases (air being particularly preferred).

The cosmetic agent according to the invention is particularly preferably produced using the following method, comprising the following steps:
i) in a stirrer vessel comprising a stirrer (and preferably a homogenizer), wax is dissolved in a liquid oil component while stirring at a temperature of at least 40° C., in particular 40° C. to 45° C.;
ii) the resulting wax solution is cooled down to a temperature of no more than 25° C.;
iii) solid matter in particulate form is dispersed in the wax solution while stirring (preferably using a homogenizer) at a temperature of no more than 25° C.;
iv) further ingredients are added while stirring at a temperature of no more than 25° C.;
v) at least one thickening agent for thickening oil, in particular silica, is added while stirring at a temperature of 20° C. to 25° C.; and
vi) a gas pressure in the stirrer vessel is applied at an overpressure in the range of 10 to 250 mbar, and the mixture is stirred for a time period of 10 to 60 minutes, in particular of 20 to 40 minutes.

A mixer such as Symex 1000 from Schröder & Boos GmbH & Co. KG, Germany, is suitable for production, for example.

It is preferred according to the invention to use, as the liquid component according to step i), one or more esters of formula (I),

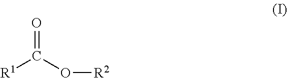

where R¹ and R², independently of one another, denote a linear or branched hydrocarbon group having 3 to 20 carbon atoms, preferably in a total amount of 1 to 20 wt. %, in particular of 5 to 15 wt. %.

It is preferred according to the invention to add at least one silicone oil (preferably in an amount of 30 to 60 wt. %, in particular of 40 to 60 wt. %) after or during step ii) (preferably between step ii) and before step iii)).

Furthermore, all preferred embodiments of the parameters of the agents according to the invention of the first subject matter of the invention are also preferred, mutatis mutandis, for the second subject matter of the invention.

A third subject matter of the invention relates to the use of a cosmetic agent of the first subject matter of the invention as a skin cosmetic.

### EXAMPLES

1.0 Production of Mousse

The following cosmetic agent according to the invention was produced:

TABLE 1
Mousse

| No. | Ingredient | Amount in wt. % |
|---|---|---|
| 1 | 2-ethylhexyl palmitate | 10.00 |
| 1 | Cocoglycerides (wax, melting point 30 to | 2.00 |
| | 33° C.) | |
| 2 | Dimethicone | to make up to 100 |
| 2 | Dimethicone crosspolymer | 5.67 |
| 2 | Cyclomethicone | 0.32 |
| 2 | Caprylic/capric triglyceride | 1.90 |
| 3 | Polyglyceryl-3-diisostearate | 1.00 |
| 3 | Titanium dioxide (CI 77891) | 4.00 |
| 3 | Iron oxides (CI 77492) | 0.45 |
| 3 | Iron oxides (CI 77491) | 0.40 |
| 3 | Iron oxides (CI 77499) | 0.12 |
| 3 | Talcum | 10.00 |
| 3 | Tapioca starch | 4.70 |
| 4 | Pentylene glycol | 0.04 |
| 4 | Water | 0.60 |
| 4 | Citric acid | 0.05 |
| 4 | Sodium carbonate | 0.05 |
| 4 | Perfume | 0.30 |
| 4 | Hydrolyzed soy protein | 0.10 |
| 4 | Spilanthes acmella flower extract | 0.05 |
| 5 | Silica (fumed) | 3.00 |

In a stirrer vessel (Symex 1000 from Schröder & Boos GmbH & Co. KG, Germany) comprising a stirrer and a homogenizer, wax was dissolved in the liquid oil component (see raw materials of Table 1 bearing the number 1) while stirring at a temperature of 43° C.

The resulting wax solution was cooled down to a temperature of no more than 25° C.

The raw materials bearing the number 2 according to Table 1 were added while stirring.

At a temperature of 25° C., the solids in particulate form bearing the number according to Table 1 were dispersed in the wax solution while stirring and additionally using a homogenizer for 20 minutes.

The ingredients bearing the number 4 according to Table 1 were added while stirring at a temperature of 25° C.

The (fumed) silica was added as a thickening agent for thickening oil while stirring at a temperature of 25° C.

Finally, the gas pressure in the stirrer vessel was increased to an overpressure of +150 mbar compared to the ambient pressure, and the mixture was stirred for a time period of 30 minutes.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent, including in each case based on the total weight of the agent:
0 to 3 wt. % free water;
one or more liquid oils in a total amount of 40 to 80 wt. %;
at least one wax;
solid matter dispersed in particulate form;
optionally further ingredients;
wherein
the cosmetic agent has a viscosity of 300000 to 800000 mPas, measured at 20° C., Brookfield, 4 revolutions per minute, spindle TE, and
the cosmetic agent has a density of 0.95 to 1.10 g/cm³ at 20° C.

2. The cosmetic agent according to claim 1, including at least one silicone oil as the liquid oil.

3. The cosmetic agent according to claim 1, including as the liquid oil at least one ester of formula (I) 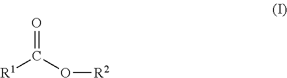
where R¹ and R², independently of one another, denote a linear or branched hydrocarbon group having 3 to 22 carbon atoms.

4. The cosmetic agent according to claim 1, wherein the wax is included in a total amount of 0.05 to 8.0 wt. %.

5. The cosmetic agent according to claim 1, wherein at least half the amount of the wax that is present is in dissolved form.

6. The cosmetic agent according to claim 1, wherein the wax includes at least one wax having a melting point of 30 to 50° C.

7. The cosmetic agent according to claim 1, wherein the solid matter dispersed in particulate form is included at a total amount of 10 to 30 wt. %.

8. The cosmetic agent according to claim 1, including at least one inorganic solid as the solid matter dispersed in particulate form.

9. The cosmetic agent according to claim 1, including at least one starch as the solid matter dispersed in particulate form.

10. The cosmetic agent according to claim 1, further including at least one thickening agent.

11. The cosmetic agent according to claim 10, wherein the thickening agent includes fumed silica.

12. The cosmetic agent according to claim 10, further including at least one emulsifier, which promotes the formation of a water-in-oil emulsion, and which is adsorbed on the solid matter dispersed in particulate form.

13. The cosmetic agent according to claim 12, wherein the at least one emulsifier adsorbed on the solid matter dispersed in particulate form includes 2 to 6 glycerol units bonding covalently to each other and at least one alkyl group having 8 to 20 carbon atoms.

14. A production method for cosmetic agents, including:
dissolving wax in a liquid oil phase forming a wax solution;
dispersing solid matter in particulate form in the wax solution;
optionally adding further ingredients to the wax solution;
optionally
a) together with the solid matter in particulate form, or
b) together with the further ingredients, or
c) separately, at the end,
adding at least one thickening agent for thickening oil, and
dispersing a gaseous substance in the resulting mixture, or during the mixing process, until the cosmetic agent has a density (measured at 20° C.) of 0.95 to 1.10 g/cm³.
